(19) United States
(12) Patent Application Publication
(10) Pub. No.: US 2018/0214454 A1
(43) Pub. Date: Aug. 2, 2018

(54) ANTIBIOTIC SOLUTION AND METHOD OF INJECTION TO PREVENT OPHTHALMIC INFECTIONS

(71) Applicant: Jason Ahee, Ivins, UT (US)

(72) Inventor: Jason Ahee, Ivins, UT (US)

(21) Appl. No.: 15/886,807

(22) Filed: Feb. 1, 2018

Related U.S. Application Data
(60) Provisional application No. 62/453,443, filed on Feb. 1, 2017.

(51) Int. Cl. A61K 31/546; A61K 31/437; A61K 38/14; A61K 9/00; A61P 27/02; A61P 31/04

(52) U.S. Cl. CPC A61K 31/546; A61K 31/437; A61K 38/14; A61P 31/04; A61K 9/0019; A61P 27/02; A61K 9/0048

(57) ABSTRACT

A method of preventing post-operative endophthalmitis involves injecting an antibiotic solution into the anterior segment of the eye during surgery, the antibiotic solution having moxifloxacin, cefuroxime, vancomycin, or some combination thereof.

### CROSS-REFERENCE TO RELATED APPLICATIONS

This application claims the benefit of U.S. Provisional Application Ser. No. 62/453,443, filed on Feb. 1, 2017, which is incorporated herein in its entirety.

### TECHNICAL FIELD

The present disclosure relates to antibiotics for the eyes. More particularly, the present disclosure relates to antibiotic solutions that are injected into the eye (i.e., intracameral) during surgery.

### BACKGROUND

Ocular surgeries are performed frequently by ophthalmologists, with special care and attention to the eye being required before, during, and after surgery. A possible complication of all intraocular surgeries is endophthalmitis, which is inflammation of the internal coats of the eye because of infection. Endophthalmitis is a serious condition that can cause blindness. Once present, a vitreoretinal specialist will usually provide an intravitreal injection of antibiotics, which may include vancomycin and ceftazidime. However, depending upon how long the eye was infected, the patient may still experience eye pain and require further surgeries. Therefore, avoiding endophthalmitis complications is preferable, with ophthalmologists using a variety of methods to lower the risk of infection.

To prevent endophthalmitis, the current standard of care includes using eye drops before and after the surgery. The eye drops are typically a combination of antibiotics, steroids, and non-steroidal anti-inflammatory medications. Normally, the drops are used 3-4 times per day for up to one month. This poses several problems including, but not limited to, potential non-compliance with the drop schedule, difficulty instilling the drops due to things like arthritis and other common physical ailments found in elderly patients, the high cost of the prescription eye drops, losing or misplacing the eye drops, and others.

Due to the problems present with eye drops, other methods have been introduced. One current method is to inject a compounded formulation (e.g., triamcinolone acetonide and moxifloxacin hydrochloride) into the posterior segment of the eye during surgery. However, properly injecting the formulation into the posterior portion of the eye is not only difficult, but increases the risk of other complications, such as zonular dehiscence, posterior pressure, and retinal toxicity or detachment, among others. For these reasons, post-operative eye drops have remained the standard.

Therefore, there remains a need for an antibiotic solution and method of delivery that reduces the risk of endophthalmitis in patients, eliminates patient error and cost to patient, and that does not require posterior injections into the eye. The present disclosure seeks to solve these and other problems.

### SUMMARY OF EXAMPLE EMBODIMENTS

In one embodiment, an antibiotic solution for injecting into an eye during an intraoperative procedure comprises 0.1% (1 mg/ml) solution of moxifloxacin.

In one embodiment, an antibiotic solution for injecting into an eye during an intraoperative procedure comprises 1% (10 mg/ml) solution of cefuroxime.

In one embodiment, an antibiotic solution for injecting into an eye during an intraoperative procedure comprises 1% (10 mg/ml) solution of vancomycin.

In one embodiment, a method of preventing post-operative endophthalmitis comprises injecting an antibiotic solution into the anterior segment of the eye during surgery, the antibiotic solution comprising moxifloxacin, cefuroxime, vancomycin, or some combination thereof.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Accordingly, the particular arrangements disclosed are meant to be illustrative only and not limiting as to the scope of the invention. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence or arrangement. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.).

As discussed in the background section, there is a need for an antibiotic solution and method of delivery that reduces the risk of endophthalmitis in patients, eliminates patient error and cost to patient, and that does not require posterior injections into the eye. As will be understood from the following description, the solutions and methods described herein solve those problems and others.

In one embodiment, an antibiotic solution for injecting into an eye during an intraoperative procedure comprises 0.1% (1 mg/ml) solution of moxifloxacin (INN—International Nonproprietary Name). Moxifloxacin has the following structure:

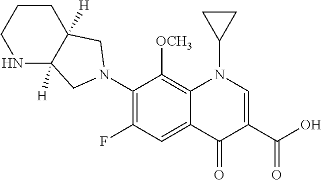 [SMILES: COc1c(N2C[C@@H]3CCCN[C@@H]3C2)c(F)cc2c(=O)c(C(=O)O)cn(C3CC3)c12]

Further details regarding the structure, preparation, and physical properties of moxifloxacin are provided in U.S. Pat. Nos. 4,990,517 and 5,607,942, and German application DE19937116, all of which are incorporated herein by reference. The antibiotic solution may be comprised of any suitable carrier fluid (e.g., liquid diluents such as water, ethyl alcohol, or propylene glycol), so long as the resulting solution has osmotic values that are compatible with the aqueous humor of the eye and ophthalmic tissues. In one embodiment, the antibiotic solution is prepared and stored in a single-dose vial, without the need for preservatives. This allows the surgeon to provide an accurate concentration in sterile environments. For example, a surgeon would inject around 0.3 milliliters of the antibiotic solution (e.g., solution comprising moxifloxacin) into the anterior segment of the eye during the intraoperative portion of cataract surgery. As a result, risk of inflammation and endophthalmitis is lowered or eliminated, without the need for the patient to self-administer antibiotic drops. Eliminating patient error significantly decreases the likelihood of endophthalmitis. Further, because the injection is into the anterior segment of the eye, the procedure is very simple for a surgeon to perform and reduces the risks of complications that can occur when injecting into the posterior segment of the eye. As such, the present method is a notable improvement over the prior art. Moxifloxacin has been used orally, intravenous, and topically on the eye. However, moxifloxacin has not been used or indicated for injections into the eye, and is marketed in the art as topical use only for the eye (e.g., eye drops). Nonetheless, beneficial—even surprising—results are achieved when injecting moxifloxacin into the anterior segment of the eye, with the risk of endophthalmitis being significantly reduced or eliminated altogether. Further, other routes of administration of moxifloxacin, or other antibiotics, for the purpose of treating endophthalmitis have proven ineffective. This is due to the blood-ocular barrier, which prevents adequate amounts of antibiotic from penetrating the eye. Accordingly, the method disclosed herein of direct injection into the anterior segment of the eye solves this problem. While the example of 0.3 milliliters was used above, it will be appreciated that the amount injected may vary, depending upon the concentration of moxifloxacin in the solution. Preferably, the antibiotic solution comprises a range of 200-600 micrograms of moxifloxacin. In the above example, where 0.3 milliliters of 0.1% (1 mg/ml) solution of moxifloxacin was injected, the total concentration of moxifloxacin injected into the eye is 300 micrograms. Nonetheless, other concentrations of moxifloxacin, from 200 micrograms to 600 micrograms, have been shown to be effective in preliminary results. Accordingly, in the examples above, the amount injected may vary depending upon the concentration of moxifloxacin and upon the dosage amount desired.

While the antibiotic solution described above contemplates the use of moxifloxacin, it will be appreciated that other antibiotics, either individually or in combination, may be used. In one embodiment, an antibiotic solution for injecting into the anterior segment of an eye during an intraoperative procedure comprises 1% (10 mg/ml) solution of cefuroxime. Cefuroxime has the following structure:

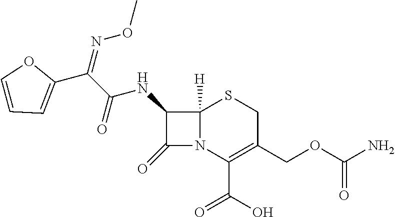 [SMILES: CO/N=C(\C(=O)N[C@@H]1C(=O)N2C(C(=O)O)=C(COC(N)=O)CS[C@H]12)c1ccco1]

In one embodiment, an antibiotic solution for injecting into the anterior segment of an eye during an intraoperative procedure comprises 1% (10 mg/ml) solution of vancomycin. Vancomycin has the following structure:

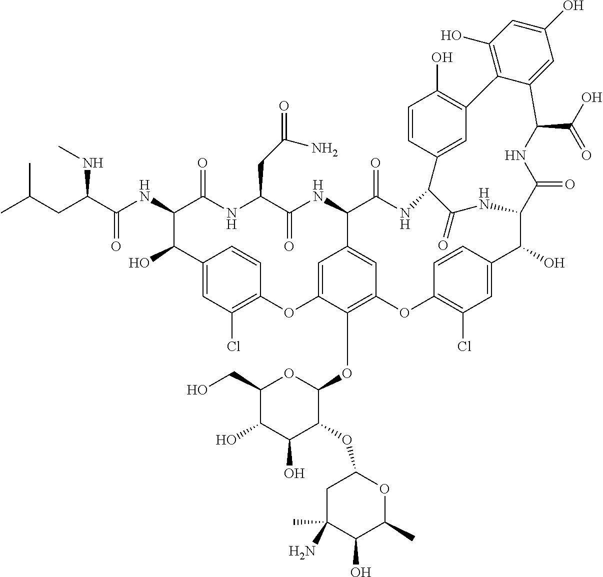 [SMILES: CN[C@H](CC(C)C)C(=O)N[C@H]1C(=O)N[C@@H](CC(N)=O)C(=O)N[C@H]2C(=O)N[C@H]3C(=O)N[C@H](C(=O)N[C@H](C(=O)O)c4cc(O)cc(O)c4-c4cc3ccc4O)[C@H](O)c3ccc(c(Cl)c3)Oc3cc2cc(c3O[C@@H]2O[C@H](CO)[C@@H](O)[C@H](O)[C@H]2O[C@H]2C[C@](C)(N)[C@H](O)[C@H](C)O2)Oc2ccc(cc2Cl)[C@H]1O]

The above-mentioned solutions are prepared in the customary manner by known methods, such as by mixing the active antibiotic or compounds with an excipient or excipients (e.g., liquid diluents such as water, ethyl alcohol, or propylene glycol). The final solution may then be sealed and stored in single-use vials known in the industry, which are preferably tinted glass, although other suitable methods and storage vials are not exempted herefrom.

In one embodiment, a method of preventing post-operative endophthalmitis comprises injecting an antibiotic solution into the anterior segment of the eye during surgery, the antibiotic solution comprising moxifloxacin, cefuroxime, vancomycin, or some combination thereof. It will be appreciated that the antibiotic solution described herein may be used on both humans and animals.

The antibiotic solutions of the present invention may also comprise one or more anti-inflammatory agents, which can be steroidal or non-steroidal. An example of steroidal anti-inflammatory agents for use herein are glucocorticoids.

As is clear from the above disclosure, the antibiotic solution and method of delivery solve the problems in the industry; namely, ease of administration by a surgeon, decreased risk of surgical complications, decreased risk of endophthalmitis, and reduced cost and burden to a patient and their insurer.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. A method of preventing post-operative endophthalmitis, comprising:
injecting an antibiotic solution into the anterior segment of an eye during eye surgery.

2. The method of preventing post-operative endophthalmitis of claim 1, wherein the antibiotic solution comprises moxifloxacin.

3. The method of preventing post-operative endophthalmitis of claim 2, wherein the solution injected into the eye comprises a range of 200 to 600 micrograms of moxifloxacin.

4. The method of preventing post-operative endophthalmitis of claim 1, wherein the antibiotic solution comprises cefuroxime.

5. The method of preventing post-operative endophthalmitis of claim 1, wherein the antibiotic solution comprises vancomycin.

6. The method of preventing post-operative endophthalmitis of claim 1, wherein the antibiotic solution comprises a combination of moxifloxacin, cefuroxime, and vancomycin.

7. The method of preventing post-operative endophthalmitis of claim 1, wherein the antibiotic solution comprises an anti-inflammatory agent.
